(19) United States
(12) Patent Application Publication
(10) Pub. No.: US 2012/0035856 A1
(43) Pub. Date: Feb. 9, 2012

(54) METHOD FOR THE DISCOVERY, VALIDATION AND CLINICAL APPLICATION OF MULTIPLEX BIOMARKER ALGORITHMS BASED ON OPTICAL, PHYSICAL AND/OR ELECTROMAGNETIC PATTERNS

(76) Inventor: Norman A. Paradis, San Marcos, CA (US)

(21) Appl. No.: 13/177,357

(22) Filed: Jul. 6, 2011

Related U.S. Application Data
(60) Provisional application No. 61/361,566, filed on Jul. 6, 2010.

(51) Int. Cl. G06F 19/10; A61B 5/145

(52) U.S. Cl. 702/19; 600/310; 600/309

(57) ABSTRACT

A method for determining multiplex biomarker algorithms based on optical, physical and/or electromagnetic patterns, and applying the multiplex biomarker algorithms so as to provide a single diagnostic result indicative of a medical condition, the method comprising:
measuring multiple physical, electromagnetic or optical patterns in the setting of experimentally induced or clinically occurring disease using at least one of physical, electromagnetic and optical sensors;
using known mathematical or machine learning algorithms to compile the measured parameters, or their signal transformed versions, into a uniplex scale or index using a clinical classifier, such that the uniplex scale or index has better clinical performance in identifying a medical condition than any of the input parameters individually;
optimizing the algorithm iteratively using additional clinical data sets and inputting patient characteristics and laboratory derived measurements;
using the uniplex scale or index to identify a medical condition; and
displaying to a user the single diagnostic result indicative of a medical condition.

### REFERENCE TO PENDING PRIOR PATENT APPLICATION

This patent application claims benefit of pending prior U.S. Provisional Patent Application Ser. No. 61/361,566, filed Jul. 6, 2010 by Norman A. Paradis for A METHOD FOR THE DISCOVERY, VALIDATION AND CLINICAL APPLICATION OF MULTIPLEX BIOMARKER ALGORITHMS BASED ON OPTICAL, PHYSICAL AND/OR ELECTROMAGNETIC PATTERNS (Attorney's Docket No. BARASH-2 PROV), which patent application is hereby incorporated herein by reference.

### FIELD OF THE INVENTION

This invention is a method for the discovery, development, validation, and clinical application of medical diagnostics based on multiplex measurement of physical, optical or electromagnetic signals from the surface of or within the body, and mathematical modeling of the same into a single useful medical biomarker algorithm.

### BACKGROUND OF THE INVENTION

The measurement of a patient's physical, chemical and anatomical properties is a central component of medical diagnosis. It has been appreciated by others that electromagnetic radiation may be directed into the body, and the transmitted or reflected energy used in medical diagnosis. The use of roentgen rays (i.e., electromagnetic radiation in the x-ray wavelengths) for production of diagnostic images is particularly well known. Also known is the transmission, absorption, and/or reflectance of near-infrared and infrared wavelengths into tissues for the measurement of various molecular species and the state of cells and tissues, such as the use of near-infrared spectroscopy to measure oxygen saturation of hemoglobin.

Recently, multiplex algorithms constructed from the measurement of multiple individual serum molecular concentrations have been widely studied as innovative diagnostics. These same approaches, however, have not been applied to non-molecular measurements such as those based on electromagnetism. Previously, medical diagnostics utilizing physical measurements have been limited to the production of images or the measurement of uniplex physiologic parameters, such as the concentration or state of single individual molecules of medical significance, usually at a single location within the body. These disparate data elements have not heretofore been combined in an attempt to achieve greater diagnostic accuracy.

### SUMMARY OF THE INVENTION

In one form of the present invention, there is provided a method for determining multiplex biomarker algorithms based on optical, physical and/or electromagnetic patterns, and applying the multiplex biomarker algorithms so as to provide a single diagnostic result indicative of a medical condition, the method comprising:

measuring multiple physical, electromagnetic or optical patterns in the setting of experimentally induced or clinically occurring disease using at least one of physical, electromagnetic and optical sensors;

using known mathematical or machine learning algorithms to compile the measured parameters, or their signal transformed versions, into a uniplex scale or index using a clinical classifier, such that the uniplex scale or index has better clinical performance in identifying a medical condition than any of the input parameters individually;

optimizing the algorithm iteratively using additional clinical data sets and inputting patient characteristics and laboratory derived measurements;

using the uniplex scale or index to identify a medical condition; and

displaying to a user the single diagnostic result indicative of a medical condition.

### DETAILED DESCRIPTION OF THE PRESENT INVENTION

The present invention is a method and apparatus for medical diagnosis based on the measurement of multiple physical signals from the surface of or within the body and the mathematical incorporation of those multiple measured signals into a single diagnostically useful medical biomarker algorithm. The medical biomarker algorithm can then be used to diagnose a medical condition in a patient.
The physical properties being measured may include, but is not limited to, electromagnetic radiation (EMR), temperature, density, weight, hydration state, transmission of sound, etc., or any combination of these inputs (i.e., data measurements).
The EMR or energy measured may be intrinsically produced within the patient's body, but a particular embodiment is based on EMR measurements derived from the transmission, and/or reflectance, and/or absorption, of EMR from an external source. The absorption and/or transmission used may be florescence, reflectance or phosphorescence. Under such circumstances, the EMR measurement would typically be numeric and the determined medical biomarker algorithm would not be an image.
The external source of EMR may be of any wavelength, but a particular embodiment is based on optical wavelengths selected for their ability to penetrate tissues and interact usefully with tissues, cells and molecular species of interest.
The signal measured may be different from, but physically coupled with, the energy transmitted into the tissues, such as the photo-acoustical effect.
The invention itself is a pattern, or transformation, of biomarkers which achieves its medical utility through mathematical construction or transformation of the measured parameters into a single synthetic clinically useful biomarker. The mathematical techniques used to create such models are well known to those familiar with the art of data mining and mathematical modeling. A particular example would be the various forms of regression, including multiple linear and logistic regressions.
The biomarker pattern or algorithm is discovered based on the use of a clinical classifier, sometimes called a “gold standard”, that allows accurate identification of patients with and without the disease.
Once classified patients' samples or data are available, the multiplex pattern or algorithm is obtained through construction or transformation of the data into mathematical model or equation.
In one particular process, the multiplex biomarker or algorithm is discovered computationally using well-known techniques of mathematical modeling and data mining.
In one embodiment of the invention, an input parameter, or pattern of the mathematical model, is used to modify the pre-test probability distribution of other inputs of the mathematical model, or even the final multiplex algorithm. By way of example but not limitation, the predictive model utilized to optimize the performance of surface temperature patterns as a diagnostic of infection or shock may be adapted as a function of different concentrations of blood lactate.
It is anticipated that measurements or patient characteristics not included in the final model or equation may modify pre-test coefficients so as to improve the diagnostic performance. Again, in a particular embodiment would be adaptation of the equation based on the results of proteomic, genomic or other in vitro diagnostic measurements.
It should be appreciated that any individual input to the mathematical model, or pattern of multiple inputs to the mathematical model, may be used in combination with any other input to create a synthetic biomarker whose diagnostic performance is superior to that of the individual inputs. It will be appreciated by those expert in the development of multiplex algorithms that such development processes may be iterative.
It should also be appreciated that the biomarker pattern or algorithm may be adaptive, improving over time or as a function of feedback within a specific epidemiologically useful unit. For example, the biomarker algorithm may be different in hospitals whose incidence of the disease is in question are different. Some of these inputs may be adaptable at the bedside, as for instance, the patient's age or sex.
Some or all of the input parameters may also be obtained internally from the patient via tomography or imaging.
The energy administered to the patient may be of a different character than the measurement used in the biomarker algorithm. In certain particular embodiments, the EMR source will be in a different location, such as in transillumination. These locations may include orifices, the gastrointestinal tract, the intravascular space, or other potential spaces.
Input data and patterns which may contribute to (i.e., be used in) the multiplex biomarker or algorithm may include, but are not limited to, the anatomic pattern, the temporal pattern, a combination of anatomic and temporal patterns and the pattern of absorbance, transmission, reflectance, florescence, phosphorescence.
The anatomic pattern includes multiple locations including axial, extremities, oral, conjunctiva, rectal, nasal, needle intra-tissue, and intravascular catheter based locations.
In a particular embodiment of the invention, the anatomic parameter of interest is depth below the skin, with different depths being used in different locations.
The temporal pattern is the change in one or more components over time.
EMR wavelengths and patterns may include optical, near infra-red spectroscopy (NIRS), Raman spectroscopy, Speckle, and surface plasmon resonance.
EMR patterns may be used in combination with non-optical data such as patient demographics, vital signs, in-vitro diagnostics, and/or any other input whose effect on the probability distribution is favorable to diagnostic performance.
Multiplex biomarker temporal and anatomic patterns may be used before and after administration of a systemic or local physical or pharmacologic agents whose physiologic effects on the probability distribution is favorable to diagnostic performance.
Adjustment of computational models may also be based on patient demographics, other vital signs, or in vitro data, among others.
A particular embodiment of the invention may incorporate administration of an affinity reagent that, when bound in vivo, has a salutary effect on the contribution of one or more input parameters to the multiplex algorithm.
In an additional specific application of the invention, both optical sensors and thermistors might be added to the standard electrocardiography leads and the pattern of temperature changes in those locations could be utilized to improve the diagnostic performance of the optical sensors.

### Implementation

In one preferred form of the present invention, the method is implemented using a computational device, e.g., an appropriately programmed general purpose computer, a dedicated computer, etc., with the output of the computational device being displayed to the user.

### EXAMPLES

Examples of situations in which a multiplex biomarker algorithm might be likely to outperform a uniplex measurement might include the following:
1. In the diagnosis of shock, sepsis, hypoxia and/or other perfusion-threatening pathologic processes:
   1. The anatomic pattern of changes in blood flow and/or oxygen status.
   2. The axial-acral distribution of measurements.
   3. The change in measurements over time.
   4. Maintenance of normal cephalic and/or central oxygenation, energetics, and/or perfusion in comparison with the extremities.
   5. Maintenance of normal deep visceral oxygenation, energetics and/or perfusion in comparison with the extremities.
   6. An algorithmic pattern incorporating anatomic, temporal, and alternative methods of sensing such as perfusion, oxygenation, and energetics.
   7. Multiplex measurement of multiple visceral organs showing sparing for the organ (such as the brain and heart) in comparison to axial musculature or skin
   8. In the resuscitation from such conditions, the above patterns, such as described above, might be expected to be reversed and could be used in evaluating the adequacy of treatment.
   9. Patterns, such as described above, may have improved diagnostic accuracy when adapted based on laboratory patterns such as in vitro diagnostic measurements of molecules related to perfusion and oxygen utilization, such as lactate.
2. In the diagnosis of regional ischemia:
   1. Comparison measurements with the contralateral region.
   2. Multiple measurements over time.
   3. Distribution of abnormal measurements demonstrating increasing ischemia, hypoxia or decreased perfusion in the more peripheral locations.
3. In using multiplex biomarker algorithms to adjust or monitor the efficacy or toxicity of medication:
   1. Anatomic patterns, such as axial-acral, superficial-deep, or visceral, may be more useful than measurements at a single location.
   2. Temporal patterns in multiplex measurements relating to the pharmacokinetics of the drug may be more useful than measurements at a single time after administration.
   3. The onset of toxicity in medications for the treatment of hypertension might be detected earlier, or more accurately, by sensing indicators of perfusion peripherally in multiple locations or over time.

### Modifications

It will be understood that many changes in the details, materials, steps and arrangements of elements, which have been herein described and illustrated in order to explain the nature of the invention, may be made by those skilled in the art without departing from the scope of the present invention.

What is claimed is:

1. A method for determining multiplex biomarker algorithms based on optical, physical and/or electromagnetic patterns, and applying the multiplex biomarker algorithms so as to provide a single diagnostic result indicative of a medical condition, the method comprising:
measuring multiple physical, electromagnetic or optical patterns in the setting of experimentally induced or clinically occurring disease using at least one of physical, electromagnetic and optical sensors;
using known mathematical or machine learning algorithms to compile the measured parameters, or their signal transformed versions, into a uniplex scale or index using a clinical classifier, such that the uniplex scale or index has better clinical performance in identifying a medical condition than any of the input parameters individually;
optimizing the algorithm iteratively using additional clinical data sets and inputting patient characteristics and laboratory derived measurements;
using the uniplex scale or index to identify a medical condition; and
displaying to a user the single diagnostic result indicative of a medical condition.

2. A method according to claim 1 wherein at least one of the physical, electromagnetic and optical sensors measures electrical potential.

3. A method according to claim 1 wherein at least one of the physical, electromagnetic and optical sensors measures photons.

4. A method according to claim 1 wherein at least one of the physical, electromagnetic and optical sensors measures physical properties such as temperature.

5. A method according to claim 1 wherein at least one of the physical, electromagnetic and optical patterns are disposed in multiple anatomic locations.

6. A method according to claim 5 wherein the physical, electromagnetic and optical patterns derived from multiple anatomic locations are utilized in the diagnostic algorithm.

7. A method according to claim 1 wherein a temporal pattern is utilized in the diagnostic algorithm.

8. A method according to claim 1 wherein both an anatomic pattern and a temporal pattern are utilized in the diagnostic algorithm.

9. A method according to claim 5 wherein the anatomic parameter of interest is depth below the skin, with different depths being used in different locations.

10. A method according to claim 1 wherein at least one of the physical, electromagnetic and optical sensors measures an electrocardiogram.

11. A method according to claim 1 wherein at least one of the physical, electromagnetic and optical sensors utilizes near-infrared spectroscopy to measure oxygen saturation of hemoglobin.

12. A method according to claim 1 wherein at least one of the physical, electromagnetic and optical sensors measures at least one of temperature, density, weight, hydration state, transmission of sound and any combination of the foregoing.

13. A method according to claim 1 wherein at least one of the electromagnetic and optical wavelengths and patterns comprise at least one of optical, near infra-red spectroscopy (NIRS), Raman spectroscopy, Speckle, and surface plasmon resonance.

14. A method according to claim 1 wherein at least one of the physical, electromagnetic and optical signals measured may be different from, but physically coupled with, the energy transmitted into the tissues, such as the photo-acoustical effect.

15. A method according to claim 1 wherein a single classifier is used to derive the algorithm.

16. A method according to claim 1 wherein multiple classifiers, each weighted differently, are used to derive the algorithm.

17. A method according to claim 16 wherein one or more of the classifiers are clinical presentations or outcomes.

18. A method according to claim 16 wherein one or more of the classifiers are laboratory derived measurements.

19. A method according to claim 1 wherein at least one of the physical, electromagnetic and optical sensors are on the surface of a patient.

20. A method according to claim 1 wherein at least one of the physical, electromagnetic and optical sensors are inside of a patient.

21. A method according to claim 1 wherein at least one of the physical, electromagnetic and optical sensors are inside of a hollow viscous organ within a patient such as the stomach, ear canal, or rectum.

22. A method according to claim 1 wherein at least one of the physical, electromagnetic and optical sensors are on the surface of a patient and one or more sensors are inside of the patient.

23. A method according to claim 1 wherein at least one of the physical, electromagnetic and optical sensors measures energy whose source is within a patient's body, preferably within the hollow viscous.

24. A method according to claim 1 wherein at least one of the physical, electromagnetic and optical sensors measures energy whose source is outside of a patient's body.

25. A method according to claim 1 wherein physical energy is directed into a patient's body to effect at least one of the physical, electromagnetic and optical patterns through transmission, absorption or reflectance.

26. A method according to claim 1 in which at least one of the physical, electromagnetic and optical patterns are used in combination with data comprising at least one of vital signs, in-vitro diagnostics, and other inputs whose effect on the probability distribution is favorable to diagnostic performance.

27. A method according to claim 1 in which at least one of patient demographics, age, and sex is included in the algorithm.

28. A method according to claim 1 in which at least one of laboratory derived data and patient demographics is used as a portion of the classifier in deriving the algorithm.

29. A method according to claim 1 in which at least one of the physical, electromagnetic and optical patterns are obtained after administration of physical or pharmacologic agents whose physiologic effects on the probability distribution is favorable to diagnostic performance.

30. A method according to claim 1 in which electromagnetic radiation (EMR) patterns are obtained before and after administration of a physical or pharmacologic agent and a change or changes in sensed physical, electromagnetic or optical patterns are components of the algorithm.
